(19) United States
(12) Patent Application Publication
(10) Pub. No.: US 2002/0115704 A1
(43) Pub. Date: Aug. 22, 2002

(54) Treatment method

(76) Inventors: Ilkka Linnankoski, Helsinki (FI); Antti Pertovaara, Helsinki (FI); Antti Haapalinna, Turku (FI); Raimo Virtanen, Rusko (FI)

Correspondence Address: FINNEGAN HENDERSON FARABOW, CARRETT & DUNNER LLP, 1300 I Street, NW, Washington, DC, 20005-3315, US

(21) Appl. No.: 10/014,573

(22) Filed: Dec. 14, 2001

(30) Foreign Application Priority Data
Dec. 15, 2000 (FI) 20002756

(51) Int. Cl. A61K 31/4164

(52) U.S. Cl. 514/396

(57) ABSTRACT

A method for the treatment of female sexual dysfunction and enhancement of normal sexual function in females with an alpha₂-adrenoceptor antagonist or a pharmaceutically acceptable ester or salt thereof.

This application claims the benefit of priority under 35 U.S.C. §119(a) to Finnish patent application no. FI 20002756, filed on Dec. 15, 2000, the contents of which are incorporated by reference herein.

An embodiment of the present invention relates to a method for the treatment of sexual dysfunction or enhancement of sexual function in a female primate with a selective alpha₂-adrenoceptor antagonist. In another embodiment, the present invention relates to a method of increasing sexual desire in a female with a specific alpha₂-adrenoceptor antagonist.

Other embodiments of the invention relate to use of a selective alpha₂-adrenoceptor antagonist for the manufacture of a medicament for the treatment of female sexual dysfunction.

Additional objects and advantages of the invention will be set forth in part in the description, which follows, and in part will be clear from the description, or may be learned by practice of the invention. The objects and advantages of the invention can be realised and attained by means of the subject matter recited in the appended claims.

### BACKGROUND OF THE INVENTION

Sex hormones (oestradiol, progesterone, testosterone) and monoaminergic neurotransmitter systems (dopamine, 5-hydroxytryptamine, noradrenaline) have modulatory effects on sexual behaviour and genital reflexes both in males and females.

A PDE5-inhibitor, sildenafil, and an unselective alpha1/alpha2-adrenoceptor antagonist, phentolamine, have been shown to increase sexual arousal i.e., erections in males. In addition, a dopamine agonist, apomorphine, has increased sexual arousal in human males. Further, an unspecific alpha₂-adrenoceptor antagonist, yohimbine, has been used for the treatment of impotence in clinical medicine (Linnankoski et al., Pharmacology Biochemistry and Behavior, Vol. 42, pp. 197-200, 1992) and a specific alpha₂-adrenoceptor antagonist, atipamezole, has been found to increase the sexual activity (ejaculations produced by copulation and/or masturbation) of males (EP 0 589 957 B).

Pharmacological modulation of sexual activity in females has been studied only sparingly. Some agents that have been effective in male erectile dysfunction have been evaluated also in females. Apomorphine and deleguamine, an alpha1/alpha2-selective alpha₂-adrenoceptor antagonist, have been shown to facilitate the sexual behaviour, lordosis response, in ovariectomised, oestrogen+progesterone-primed female rats (Hamburger-Bar, R. and Rigter, H. (1975) European Journal of Pharmacology 32: 357-360; Conzales et al., European Journal of Pharmacology 312, (1996) 1-6). In a recent study, a mild improvement in the sexual arousal of menopausal women, such as self-reported lubrication, with oral phentolamine has been reported (Rosen R. C., et al. (1999) Journal of Sex and Marital Therapy 25 (2): 137-144). However, in women with hypoactive sexual desire, yohimbine was found to be without any therapeutic effect on sexual desire (Piletz J. E., et al. (1998) Journal of Sex and Marital Therapy 24 (1):43-54). Further, sildenafil did not improve the sexual function significantly in postmenopausal women (Kaplan, S. A., et al. (1999) Urology 53(3):481-6). Thus, compounds having an effect in sexual behaviour in males, especially on erectile dysfunction, or modulated lordosis behaviour in oestrogen+progesterone-primed female rats, are not self-evidently effective for the treatment of female sexual dysfunction.

The sexual behaviour of females to a large extent, at least in animal studies, can be assessed indirectly from the behaviour of the female together with a male (Linnankoski, I. and Leinonen, L. (1985) Z. Tierpsychol., 70:115-122.). Sexual behaviour of the stump-tailed macaque Macaca arctoides provides an experimental animal model that resembles human sexual behaviour more than that of many other non-human primates. The sexual activity of Macaca arctoides, like that of humans and unlike that of many other non-human primates and other animal species (rats, dogs ect.), is only slightly influenced by season and menstrual cycle (Linnankoski, I., et al. (1981) Arch. Sexual Behav. 10:207-222). Although with Macaca arctoides as with humans the male is the active partner in sexual behaviour, the female is able to trigger the sexual activity of the male by various psychosocial gestures such as eye contacts or exposure of perineal region to the male (Linnankoski, I., et al. (1993) Folia Primatol. 60:181-184). Therefore, under appropriate experimental conditions the observation of the male sexual activity triggered by the female can be used as an indirect measure of female sexual activity.

### BRIEF DESCRIPTION OF THE DRAWINGS

FIG. 1 illustrates the effects of the administration of atipamezole (Atipam), apomorphine (Apomor) or their combination (Ati+Apo) in the female monkey on sexual behaviour of the male with the female. A) Copulations with ejaculation. B) Copulations without ejaculation. C) All forms of copulations. D) Short-time mountings. The error bars represent S.E.M. (n=4). * p<0.05 (Tukey's test; reference: the Saline condition=Sal). The doses of atipamezole and apomorphine alone are shown below bars in mg/kg. Ati+Apo_{low}=atipamezole 0.03 mg/kg+apomorphine 0.125 mg/kg, Ati+Apo_{high}=atipamezole 0.3 mg/kg+apomorphine 0.25 mg/kg.

### DETAILED DESCRIPTION OF THE INVENTION

Applicants have surprisingly discovered that a specific alpha₂-adrenoceptor antagonist, atipamezole, increases sexual desire and arousal in female primates, whereas the dopamine agonist, apomorphine, is ineffective.

Applicants' investigations show that administration of a specific alpha₂-adrenoceptor antagonist, atipamezole, to a female primate increased sexual behaviour of the male with the female. This finding indicates that atipamezole increased sexual desire and arousal in the female, and this change in psychosocial behaviour of the female triggered the increased sexual activity of the male. Apomorphine alone or in combination with atipamezole did not induce any significant changes in the sexual behaviour of the female monkeys.

Thus, specific alpha₂-adrenoceptor antagonists such as atipamezole, and their pharmacologically acceptable esters or salts, can be used for the treatment of female sexual dysfunction. In addition, they can be used in the manufacture of a medicament for the treatment of female sexual dysfunction.

Female sexual dysfunction includes decreased sexual desire, decreased sexual arousal, dyspareunia and diminished ability to achieve orgasm. Sexual dysfunction may be caused directly or indirectly by several diseases or conditions, such as various somatic (e.g. neurological or vascular) diseases, and psychiatric disorders. Psychiatric disorders include, but are not limited to, affective disorders, such as depression or anxiety disorders, such as Generalized Anxiety Disorder (GAD), Social Anxiety Disorder (SAD), Panic Disorder (PD), Obsessive Compulsive Disorder (OCD) and Post-Traumatic Stress Disorder (PTSD). In addition, various drugs used for the treatment of psychiatric disorders, for example anxiolytic compounds like buspirone and benzodiazepines such as diazepam, alprazolam and lorazepam, antidepresssants such as SNRIs (e.g. venlafaxine) and SSRIs, frequently cause sexual disorders such as decreased libido as an adverse effect.

In the present invention, female primates regardless of their hormonal status, i.e. sexual mature female primates from premenopausal period up to postmenopausal age, are included. For the purposes of this disclosure and claims the term “premenopausal period” relates to the time of sexual maturity preceding menopause, and the term “postmenopausal age” relates to time after menopause. The term “treatment” means treatment in order to remedy or alleviate the disorder or its symptoms, and treatment in order to prevent the development or the exacerbation of the disorder or its symptoms. In addition to treating female sexual dysfunction, the compounds of the invention may be used on-demand to enhance sexual function in females, who are considered to have normal sexual function.

Atipamezole and its structural analogues provide substantial advantages over the compounds that have been earlier suggested to be useful in the treatment of sexual dysfunction. Their ability to improve sexual desire plays an important role in the treatment of female sexual dysfunction. Female sexual dysfunction is a complicated disorder comprising several symptoms of which decreased or lacking desire is the primary problem that causes e.g. arousal and pain disorders and are likely the main cause of orgasm disorders as well. Lack of desire can also cause cognitive distraction during the intercourse. Distraction during intercourse has been shown to be increased in women having lower sexual esteem, less sexual satisfaction, and less consistent orgasms. The compounds of the invention also prevent female sexual dysfunction caused by cognitive distraction during sexual activity with a partner leading to lowered sexual esteem and sexual satisfaction and less consistent orgasms.

Furthermore, the compounds of the invention are substantially void of side effects connected to previously known compounds that have been used in the treatment of sexual dysfunction. For instance, they have no significant effect at therapeutic doses on cardiovascular functions, do not cause significant sedation, headache, nausea and vomiting and dizziness usually seen with compounds having effects on either dopaminergic or 5-hydroxytryptaminergic (5-HT) system. Furthermore, they will not cause significant motor dysfunctions (dyskinesias, dystonia, rigidity), hallusinations or psychotic effects usually connected with compounds having effect on dopaminergic system. Moreover, they do not cause significant dependence and/or abuse liability usually seen with compounds having effect on either dopaminergic or 5-hydroxytryptaminergic (5-HT) or glutaminergic system.

The compounds of the invention do not significantly interfere with the hormonal system. Consequently, they do not induce breast or endometrium cancer involved with hormonal treatment and they do not cause hirsutism connected to the use of androgenic compounds. Furthermore, the compounds, such as atipamezole, a halogenated analogue of atipamezole, or a pharmaceutically acceptable salt thereof, can be administered so as not to unduly interfere with sleep of the female after the administration and subsequent sexual activity. For example, when taken on demand in the evening, the effects of the compounds do not significantly interfere with the ability of the female to sleep through the night.

The compounds of the invention may also be used to treat female sexual dysfunction caused by other medicaments, such as antipsychotics and antidepressants. Antipsychotics agents to be used include, but are not limited to haloperidol, clotzapine, risperidone, sertindole, flupentixole. Antidepressants of the present invention include, without limitation, tricyclic antidepressants, such as clompramine, mianserine, amitriptyline and nortriptyline, monoamine oxidases, including MAO-A inhibitors, such as moclobemide, SSRIs such as fluoxetine, paroxetine, sertraline and citalopram, or SNRIs such as venlafaxine.

The compounds of the invention may be used in conjunction with other compounds that are useful in the treatment of sexual dysfunction, especially with those that are useful in the treatment of decreased sexual arousal, dyspareunia and diminished ability to achieve orgasm. These other compounds include, but are not limited to, vascular smooth muscle relaxants, such as PDE5 inhibitors (e.g. silfenadil and alprostadil) or local vasodilators such as nitroglyserine products.

The compounds of the invention include selective alpha₂-adrenoceptor antagonists. For example, the compound can be a specific alpha₂-adrenoceptor antagonist having insignificant affinity for other neurotransmitter receptors than alpha₂-adrenoceptors

For the purposes of this disclosure and claims the term “specific alpha₂adrenoceptor antagonist” refers to a compound having no other major receptor affinities than alpha₂-adrenoceptor affinity, for example no direct dopaminergic and serotonergic effects (such as 5-HT1A or 5-HT1D) affinity. Further, the specificity in relation both to alpha₂/alpha₁ and alpha₂/5-HT1A can be over 300 and 1100 selectivity rations, respectively.

Specific alpha₂-adrenoceptor antagonists include, without limitation, atipamezole (4-(2-ethyl-2,3-dihydro-1H-inden-2-yl)-1H-imidazole), and its pharmaceutically acceptable acid addition salts with inorganic and organic acids or prodrugs generally used for the purpose, and the halogenated analogues of atipamezole, such as 4-(2-ethyl-5-fluoro-2,3-dihydro-1H-inden-2-yl)-1H-imidazole and 4-(2-ethyl-5,6-difluoro-2,3-dihydro-1H-inden-2-yl)-1H-imidazole and their pharmaceutically acceptable acid addition salts. These compounds are described in U.S. Pat. No. 4,689,339 and U.S. Pat. No. 5,498,623, respectively, the contents of which are incorporated by reference herein.

Atipamezole is a potent α₂-adrenoceptor antagonist which has a high α₂-/α₁-adrenoceptor selectivity ratio. Unlike various other α₂-adrenoceptor antagonists, it has negligible affinity for any other neurotransmitter receptors such as dopaminergic, GABAergic, serotonergic (such as 5-HT_{1A}) ect. receptors, thus being also a specific α₂-adrenoceptor antagonists. The specificity and selectivity of various other known α₂-adrenoceptor antagonist have been questioned. Yohimbine has affinity also to various other than noradrenergic receptors such as dopaminergic, 5-hydroxytryptaminergic receptors and benzodiazepine receptors. Idazoxan and also various more α₂-/α₁-selective α₂-adrenoceptor antagonists such as RX821002, (2-methoxy idazoxan), delequamine (RS15385), BRL 44408 and ARC 239 have affinity also on 5-hydroxytryptamine (5-HT) 5-HT_{1A} receptors or 5-HT1D receptors, thus being less α₂-adrenoceptor/5-HT receptor selective than atipamezole.

The precise amount of the drug to be administered to a primate according to the present invention can depend on numerous factors known to one skilled in the art, such as the compound to be administered, the general condition of the patient, the condition to be treated, the desired duration of use, the type of primate, the method of administration etc. For example, suitable daily dosage for a female having normal weight (65 kg-70 kg) is 1-50 mg. An example time period for administration is about 0.5 to 2 hours before sexual activity.

The dosages of other medicaments to be used in combination with the compounds of the invention may be chosen according to principles generally known to the medical practitioners, i.e. the usual recommended oral daily dosages may be used.

Typical routes of administration include, without limitation, oral, transdermal, transmucosal, and parenteral routes. One skilled in the art would recognise the dosage forms suitable in the method of the present invention.

The compounds of the invention may be used, for example, on-demand to treat a sexual disfunction or to enhance sexual function in the female primate. For instance, the compounds may be administered shortly before planned sexual activity. The compounds may also be administered regularly on a routine schedule to maintain a certain level of, for example, sexual desire or sexual arousal. Such maintained level of sexual desire or sexual arousal may be, for example, an objectively normal or enhanced level for a female primate who otherwise suffers from a sexual disfunction, or an enhanced level for a female primate not suffering from a sexual disfunction.

The present invention provides a new solution in the pharmacotherapy of female sexual dysfunction. It will be further clarified by the following example, which is intended to be purely exemplary of the invention.

### EXAMPLE

This example investigated whether the administration a specific alpha₂-adrenoceptor antagonist, atipamezole, a dopamine agonist, apomorphine, or their combination into a female Macaca arctoides produces changes in the sexual behaviour of the female with a male.

The experiments were performed using four female and two male Macaca arctoides monkeys born in captivity. The ages of the adult females were 13 and 16 years and those of the aged females 23 and 25 years. The ages of the males were 14 and 23 years. The females were sexually mature and with normal hormonal cycles. There were no signs of pregnancy during the experiments.

During the testing period, the monkeys were housed individually in stainless-steel cages. They were fed commercially available food twice each day, and water was always available. Females were weighed before the first injection of each series and doses were adjusted accordingly when appropriate. Sexual behaviour was tested between 11.00 and 16.00 h. The same test cage and testing procedure were used in all experiments. One experienced observer, blind to the treatment, viewed the animals at a distance of about 0.5 m from the cage.

During the testing period, the couple being tested was housed in a single case (0.6×0.9×1.2 m) with two compartments. During the first 10 min of each session, a sliding wall made of steel bars separated the male and the female in the test cage. The monkeys could see and touch each other through this wall. Between the testing sessions, the female being tested was housed in another room with no visual contact with the male.

After i.m. administration of the studied compounds to the female, the observation of the sexual behaviour began as described subsequently. Ten minutes after the end of drug administration, the sliding wall between the male and the female was pulled away and the observation of sexual activity continued for the next 20 min. At the end of the observation period, the sliding wall was replaced and the female was taken to her home cage in another room.

The occurrence and duration of the following parameters were observed: perineal investigation, mounting, masturbation, ejaculation, male and female grooming, eye contact, tying, yawning, self-scratching, direct male aggression towards the female and teeth grinding. A single mount leading to ejaculation was counted as ejaculation. A single mount not leading to ejaculation was counted as a short term mount. If the interval between mounts was >30 s, the mount was considered to be separate. In Macaca arctoides, male ejaculation reached in copulation or masturbation can be recognised clearly on the basis of its stereotypic manifestations as described in Linnankoski et al. (Arch. Sexual Behav. 10:207-222, 1981).

The experiments were performed once every other day for three days/week. In each female, the effect of atipamezole (Orion Pharma, Turku, Finland) alone (0.03 and 0.3 mg/kg and saline control) was tested during the first two weeks. The effect of apomorphine (Sigma, St.Louis, Mo.) alone (0.125 and 0.25 mg/kg and saline control) was tested during the next two weeks. The combination of a low dose of atipamezole (0.03 mg/kg) with a low dose apomorphine (0.125 mg/kg), a high dose of atipamezole (0.3 mg/kg) with a high dose of apomorphine (0.25 mg/kg), or saline control (two saline injections) was studied during the last two weeks. Owing to the difference in their pharmacokinetic properties, atipamezole (or its saline control) was always administered 10 min before testing session and apomorphine (or its saline control) 20 min before the testing session. The order of testing various doses of drugs and saline control was randomised within each two week testing period. Each dose or the combination was tested twice and the average result of these two sessions was used in final calculations. The experiments were performed in a double-blind fashion.

The statistical evaluation of the data was performed using one-way analysis of variance with repeated measures followed by Tukey-Kramer test. Effect of each compound was assessed separately. P<0.05 was considered to represent a significant difference.

Results

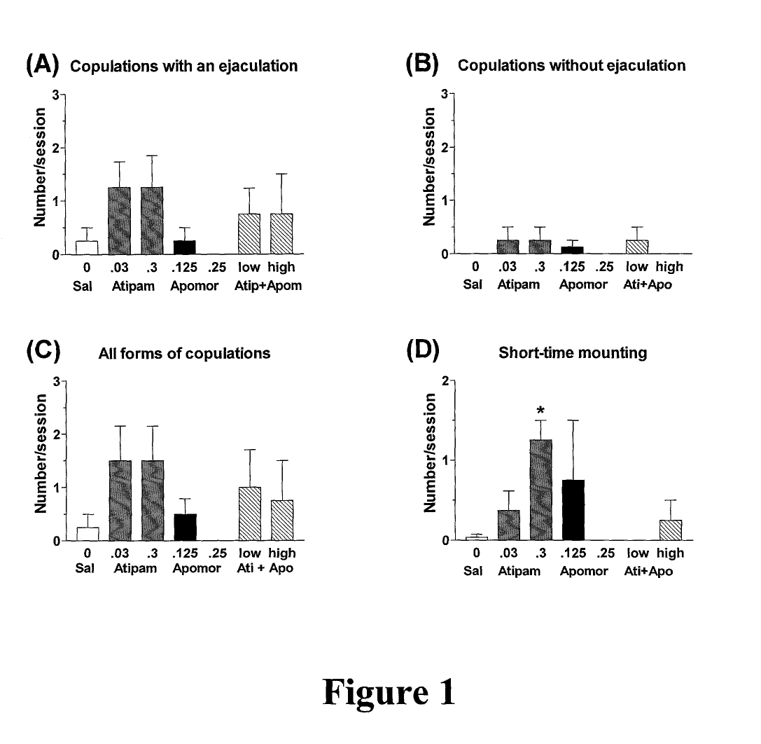

When copulations with ejaculation and those without ejaculation were assessed separately, the effects of atipamezole, apomorphine or their combinations failed to be statistically significant. The results are presented in FIGS. 1A and 1B. When all the copulations (with and without ejaculation) were pooled, atipamezole administered in the females produced a significant increase in the number of copulations (F_{2,11}=5.00, p=0.05). The result is presented in FIG. 1C. Effects of apomorphine alone or together with atipamezole on the total number of copulations were not significant. Atipamezole produced a dose-dependent increase in the number of short time mounts (F_{2,11}=7.55, p<0.03) The result is presented in FIG. 1D. Apomorphine alone or in combination with atipamezole had no significant effect on short time mounting activity. The effects of the compounds did not vary with the menstrual cycle.

Although the invention has been illustrated by the preceding example, it is not to be construed as being limited to the materials employed therein; rather, the invention is directed to the generic area as herein disclosed. Various modifications and embodiments thereof can be made without departing from the spirit or scope thereof.

We claim:

1. A method for the treatment of female sexual disfunction or for the enhancement of female sexual function in a female primate, which comprises administering to the female primate in need thereof an effective amount of at least one selective alpha₂-adrenoceptor antagonist.

2. The method according to claim 1, wherein the at least one alpha₂-adrenoceptor antagonist is a specific alpha₂-adrenoceptor antagonist.

3. The method according to claim 2, wherein the specific alpha₂-adrenoceptor antagonist is atipamezole, a halogenated analogue of atipamezole, or a pharmaceutically acceptable salt thereof.

4. The method according to claim 1, which comprises the treatment of female sexual disfunction.

5. The method according to claim 1, which comprises the treatment of female sexual disfunction, wherein the female sexual disfunction is impaired sexual desire.

6. The method according to claim 1, which comprises the treatment of female sexual disfunction, wherein the female sexual disfunction is impaired sexual arousal.

7. The method according to claim 1, which comprises the treatment of female sexual disfunction, wherein the female sexual disfunction is dyspareunia.

8. The method according to claim 1, which comprises the treatment of female sexual disfunction, wherein the female sexual disfunction is diminished ability to achieve orgasm.

9. The method according to claim 1, which comprises the treatment of female sexual disfunction, wherein the female sexual disfunction is caused by cognitive distraction during sexual activity with a partner leading to lowered sexual esteem and sexual satisfaction and less consistent orgasms.

10. The method according to claim 1, wherein the primate is a human.

11. The method according to claim 1, which comprises administering the at least one alpha₂₋adrenoceptor antagonist about 0.5 to about 2 hours before sexual activity.

12. The method according to claim 1, which further comprises administering at least one antipsychotic agent with the at least one alpha₂₋adrenoceptor antagonist.

13. The method according to claim 1, which further comprises administering at least one antidepressant with the at least one alpha₂₋adrenoceptor antagonist.

14. The method according to claim 1, which further comprises administering at least one vascular smooth muscle relaxating agent with the at least one alpha₂₋adrenoceptor antagonist.

15. The method according to claim 14, wherein the at least one vascular smooth muscle relaxating agent is a PDE5 inhibitor or nitroglycerin.

16. A method for the enhancement of sexual function in a female primate, which comprises administering to the female primate an effective amount of atipamezole, a halogenated analogue of atipamezole, or a pharmaceutically acceptable salt thereof.

17. A method as claimed in claim 16, wherein the primate is a human.
